(19) United States
(12) Patent Application Publication
(10) Pub. No.: US 2026/0151600 A1
(43) Pub. Date: Jun. 4, 2026

(54) INTEGRATED CATHETER ASSEMBLY HAVING A GUIDEWIRE REDIRECTION COMPONENT

(71) Applicant: Becton, Dickinson and Company, Franklin Lakes, NJ (US)

(72) Inventors: Adam John Boud, Bluffdale, UT (US); Tyler Warner, Bluffdale, UT (US); Chad Alan Tagge, Sandy, UT (US)

(21) Appl. No.: 19/359,484

(22) Filed: Oct. 15, 2025

Related U.S. Application Data
(60) Provisional application No. 63/727,075, filed on Dec. 2, 2024.

(51) Int. Cl. A61M 25/09; A61B 5/15; A61M 25/00; A61M 39/10

(52) U.S. Cl. CPC A61M 25/09041; A61B 5/150267; A61B 5/150992; A61M 25/0009; A61M 39/10; A61M 2039/1077

(57) ABSTRACT

The catheter assembly may include a catheter adapter and a catheter extending from a distal end of the catheter adapter. A lumen of the catheter and a lumen of the catheter adapter may include an open pathway extending along an axis and configured to receive a guidewire when the catheter assembly is threaded distally over the guidewire. The catheter assembly may include a redirection component having a surface disposed at a proximal end of the open pathway to block the open pathway. The surface may be configured to redirect the guidewire from the axis into a side port of the catheter assembly. The surface may be angled with respect to the axis. The surface may be planar or curved. A method of manufacturing may include providing the catheter adapter, coupling the catheter to the catheter adapter, and positioning a redirection component within the lumen of the catheter adapter.

### RELATED APPLICATIONS

This application claims the benefit of United States Provisional Patent Application No. 63/727,075, filed December 2, 2024, and entitled INTEGRATED CATHETER ASSEMBLY HAVING A GUIDEWIRE REDIRECTION COMPONENT, which is incorporated herein in its entirety.

### BACKGROUND

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter device includes a catheter that is over-the-needle. As its name implies, the catheter that is over-the-needle may be mounted over an introducer needle having a sharp distal tip. The catheter and the introducer needle are generally inserted into a blood vessel at a shallow angle through the skin into vasculature of the patient. After the clinician confirms the catheter is within the blood vessel, the introducer needle is removed and the catheter is left in place for future infusion and/or blood draw.

One problem with the catheter that is mounted over-the-needle is that it is possible to get a blood flash when the introducer needle enters a blood vessel but the catheter is still outside the blood vessel. This is because there is an offset between the tip of the introducer needle and the start of the catheter that is over-the-needle. The offset between the tip of the introducer needle and the start of the catheter can lead to inappropriate placement of the catheter and failed intravenous (IV) attempts when the clinician tries to advance the catheter when the catheter is partially or completely outside the blood vessel. A potential way to prevent this from happening is inserting a guidewire through a needle inserted into the blood vessel, removing the needle, and then advancing the catheter over the guidewire (Seldinger technique).

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to vascular access devices, systems, and methods. In particular, the present disclosure relates to a catheter assembly having a redirection component configured to redirect a guidewire into a side port of the catheter assembly. Thus, in some embodiments, the catheter assembly of the present disclosure improves use of the Seldinger technique or similar technique with a catheter assembly that includes a catheter adapter having a side port.

In some embodiments, a catheter assembly may include a catheter adapter, which may include a distal end, a proximal end, an inner surface forming a lumen extending between the distal end of the catheter adapter and the proximal end of the catheter adapter, and a side port disposed between the distal end of the catheter adapter and the proximal end of the catheter adapter.

In some embodiments, the catheter assembly may include a catheter extending from the distal end of the catheter adapter. In some embodiments, the catheter may include a distal end, a proximal end, and a lumen extending between the distal end of the catheter and the proximal end of the catheter. In some embodiments, the lumen of the catheter and the lumen of the catheter adapter may include an open pathway, which may extend along an axis. In some embodiments, the open pathway may be configured to receive a guidewire when the catheter assembly is threaded distally over the guidewire.

In some embodiments, the catheter assembly may include a redirection component disposed within the lumen of the catheter adapter and sealing the lumen of the catheter adapter proximal to the side port. In some embodiments, the redirection component may include a distal end having a surface disposed at a proximal end of the open pathway to block the open pathway. Thus, in some embodiments, the open pathway may extend along the axis to the surface or to a portion of the surface aligned with the axis. In some embodiments, the surface may be configured to redirect the guidewire from the axis into the side port when the catheter assembly is threaded distally over the guidewire. In some embodiments, the side port may be angled with respect to the axis.

In some embodiments, the surface may include a groove, which may include a rounded trough shape. In these and other embodiments, a cross-section of the groove may include a linear portion angled with respect to the axis. In these embodiments, the surface may be angled with respect to the axis and planar. In some embodiments, the surface may be curved or arc shaped.

In some embodiments, the catheter assembly may include a canister disposed within the lumen of the catheter adapter. In some embodiments, the redirection component may be secured within the canister. In some embodiments, the canister may be secured to the catheter adapter. In some embodiments, the redirection component may be constructed of a first material and the canister may be constructed of a second material. In some embodiments, the first material may be less rigid than the second material.

In some embodiments, the side port may be angled between 45˚ and 90˚, inclusive, with respect to the axis.

In some embodiments, the redirection component may permanently seal the lumen of the catheter adapter proximal to the side port.

In some embodiments, the inner surface of the catheter adapter may include an annular groove. In some embodiments, the redirection component may include an outer surface having an annular protrusion configured to lock the redirection component within the annular groove.

In some embodiments, the side port may include an inner surface forming a side port lumen. In some embodiments, the surface may be distal to or aligned with a proximal wall of the inner surface of the side port.

In some embodiments, the side port may include the inner surface forming a side port lumen, and the distal end of the redirection component may include a first opening and a second opening separated from the first opening by an arm. In some embodiments, when the catheter assembly is threaded distally over the guidewire, the first opening may move over the guidewire before the second opening. In some embodiments, a central axis of the first opening may be aligned with the axis. In some embodiments, a proximal end of the second opening may be distal to or aligned with a proximal wall of the inner surface of the side port.

In some embodiments, the catheter assembly may include a wedge disposed within the lumen of the catheter adapter and securing the catheter within the catheter adapter. In some embodiments, the open pathway may extend through the wedge.

In some embodiments, a method of manufacturing may include providing the catheter adapter. In some embodiments, the method of manufacturing may include coupling the catheter to the catheter adapter. In some embodiments, the method of manufacturing may include positioning the redirection component within the lumen of the catheter adapter.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present disclosure. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A is a schematic diagram of the Seldinger technique of the prior art, illustrating insertion of a needle through the skin into a blood vessel;

FIG. 1B is a schematic diagram of the Seldinger technique of the prior art, illustrating a guidewire passed through the needle into a lumen of the blood vessel;

FIG. 1C is a schematic diagram of the Seldinger technique of the prior art, illustrating the needle withdrawn, leaving the guidewire in the lumen of the blood vessel;

FIG. 1D is a schematic diagram of the Seldinger technique of the prior art, illustrating a catheter threaded over the guidewire and passed into the lumen of the blood vessel;

FIG. 1E is a schematic diagram of the Seldinger technique of the prior art, illustrating the guidewire withdrawn leaving the catheter in position in the lumen of the blood vessel;

FIG. 1F is a cross-sectional view of a prior art catheter assembly;

FIG. 2A is an upper perspective view of an example catheter assembly, illustrating an example redirection component having an example surface redirecting an example guidewire from an example axis into an example side port when the catheter assembly is threaded distally over the guidewire, according to some embodiments;

FIG. 2B is an upper perspective view of an example guidewire, according to some embodiments;

FIG. 2C is an upper perspective view of the redirection component of FIG. 2A, according to some embodiments;

FIG. 2D is an upper perspective view of the redirection component of the FIG. 2A, illustrating the surface redirecting the guidewire from the axis into the side port, a catheter adapter and the catheter being removed for illustrative purposes, according to some embodiments;

FIG. 2E is a cross-sectional view of the catheter assembly of FIG. 2A, illustrating the surface of the redirection component redirecting the guidewire from the axis into the side port when the catheter assembly is threaded distally over the guidewire, according to some embodiments;

FIG. 2F is a cross-sectional view of the catheter assembly of FIG. 2A, illustrating the surface of the redirection component, according to some embodiments;

FIG. 3A is an upper perspective view of another example catheter assembly having an example canister and another example redirection component, illustrating the redirection component having the surface contacting the guidewire disposed on the axis just prior to redirection of the guidewire by the surface when the catheter assembly is further threaded distally over the guidewire, according to some embodiments;

FIG. 3B is an upper perspective view of the canister of FIG. 3A, according to some embodiments;

FIG. 3C is another upper perspective view of the canister of FIG. 3A, according to some embodiments;

FIG. 3D is a side view of the redirection component of FIG. 3A, according to some embodiments;

FIG. 3E is another side view of the redirection component of FIG. 3A, opposite the side view of FIG. 3D, according to some embodiments;

FIG. 3F is an upper perspective view of the redirection component of FIG. 3A, according to some embodiments;

FIG. 3G is a cross-sectional view of the redirection component of FIG. 3A, according to some embodiments;

FIG. 3H is a cross-sectional view of the catheter assembly of FIG. 3A, illustrating the surface of the redirection component redirecting the guidewire from the axis into the side port when the catheter assembly is threaded distally over the guidewire, according to some embodiments;

FIG. 3I is a cross-sectional view of the catheter assembly of FIG. 3A, illustrating the surface of the redirection component, according to some embodiments;

FIG. 4A is an upper perspective view of another example surface of another example redirection component, the surface being angled with respect to the axis and planar, according to some embodiments;

FIG. 4B is a top view of the surface of the redirection component of FIG. 4A, according to some embodiments;

FIG. 4C is a cross-sectional view of the redirection component of FIG. 4A, according to some embodiments;

FIG. 5A is an upper perspective view of another example surface of another example redirection component, the surface being arced or curved, according to some embodiments; and

FIG. 5B is a cross-sectional view of the surface of the redirection component of FIG. 4A, according to some embodiments.

### DESCRIPTION OF EMBODIMENTS

For clarity it is to be understood that the word “proximal” refers to a direction relatively closer to a clinician using the catheter assembly to be described herein, while the word “distal” refers to a direction relatively further from the clinician. For example, the end of a catheter placed within the body of a patient is considered a distal end of the catheter, while the catheter end remaining outside the body is a proximal end of the catheter. Also, the words “including,” “has,” and “having,” as used in the present disclosure, including the claims, shall have the same meaning as the word “comprising.”

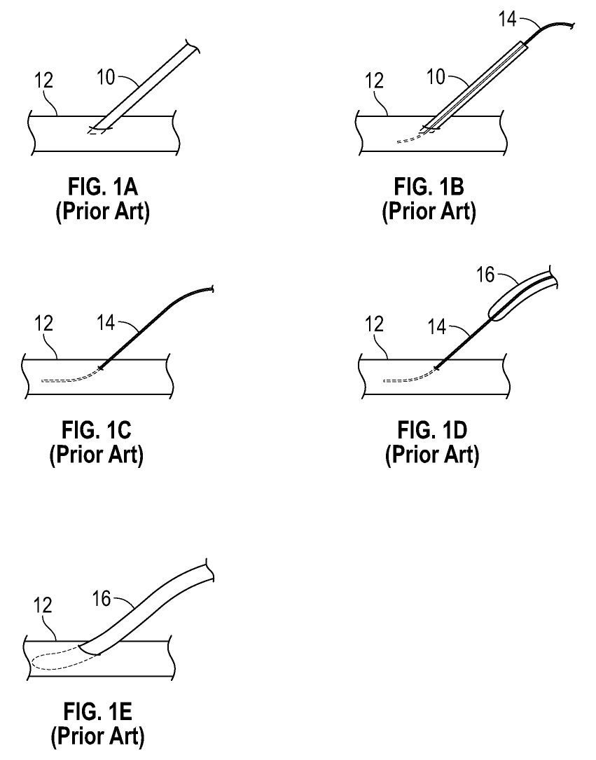

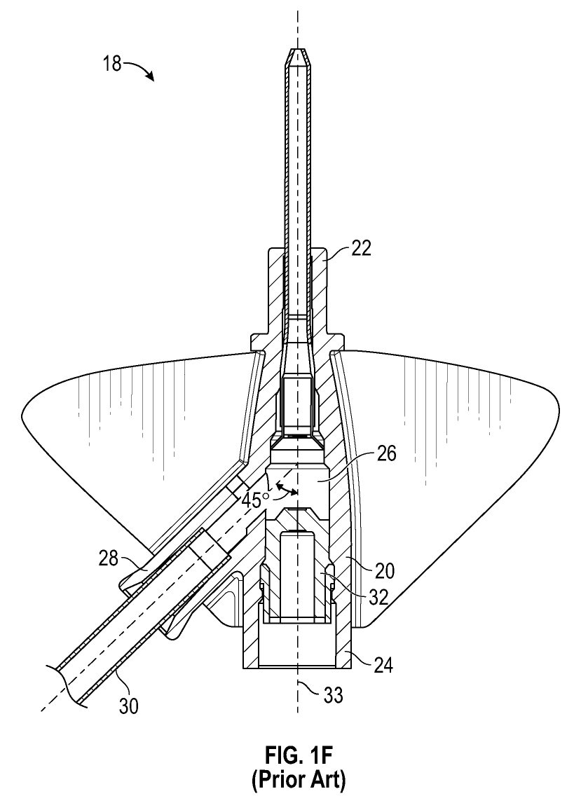

Referring now to FIGS. 1A-1E, steps of the Seldinger technique are illustrated in sequential order. First, as illustrated in FIG. 1A, a needle 10 having a sharp tip is inserted through the skin of a patient into a lumen of a blood vessel 12 of the patient. After the needle 10 is inserted through the skin of the patient into the blood vessel 12, a guidewire 14 is passed through the needle 10 into the lumen of the blood vessel 12, as illustrated in FIG. 1B. After the guidewire 14 is passed through the needle 10 into the lumen of the blood vessel 12, the needle 10 is withdrawn leaving the guidewire 14 in the lumen of the blood vessel 12, as illustrated in FIG. 1C. After the needle 10 is withdrawn leaving the guidewire 14 in the lumen of the blood vessel 12, a catheter 16 is threaded over the guidewire 14 and passed into the lumen of the blood vessel 12, as illustrated in FIG. 1D. After the catheter 16 is threaded over the guidewire 14 and passed into the lumen of the blood vessel 12, the guidewire 14 is withdrawn leaving the catheter 16 in position in the lumen of the blood vessel 12.

Referring now to FIG. 1F, a prior art catheter assembly 18 is illustrated. The prior art catheter assembly 18 includes a catheter adapter 20 having a distal end 22, a proximal end 24, and a lumen 26 extending through the distal end 22 and the proximal end 24. A catheter 27 extends from the distal end 22 of the catheter adapter. The catheter 27 is over-the-needle and an introducer needle (not shown) extends through the catheter 27 to aid insertion of the catheter 27 into vasculature of a patient. The prior art catheter assembly 18 may correspond to the BD NEXIVA™ Closed IV Catheter System available from Becton Dickinson & Company of Franklin Lakes, New Jersey.

The prior art catheter assembly 18 is an integrated intravenous (IV) catheter assembly. An integrated IV catheter assembly is an IV catheter assembly having an integrated extension set. Such extension sets typically include an extension tubing 30 having one end integrated into the catheter adapter and another end including an access port. Here, the prior art catheter assembly 18 includes a side port 28 and the extension tubing 30 extending from the side port 28.

Integrated IV catheter assemblies are oftentimes used to draw blood. For example, after inserting the catheter 27 into vasculature of a patient, a clinician may allow blood to flow into the extension set proximally to the access port. Once the blood has flowed proximally to the access port, the clinician may attach a blood collection set (e.g. a vacuum tube adapter) to the access port to collect a blood sample.

A septum 32 constructed of an elastomeric material is disposed within the lumen 26 of the catheter adapter 20 and prevents blood from leaking out the proximal end 24 of the catheter adapter 20. The side port 28 is disposed at a 45˚ angle with respect to a longitudinal axis 33 of the prior art catheter assembly 18.

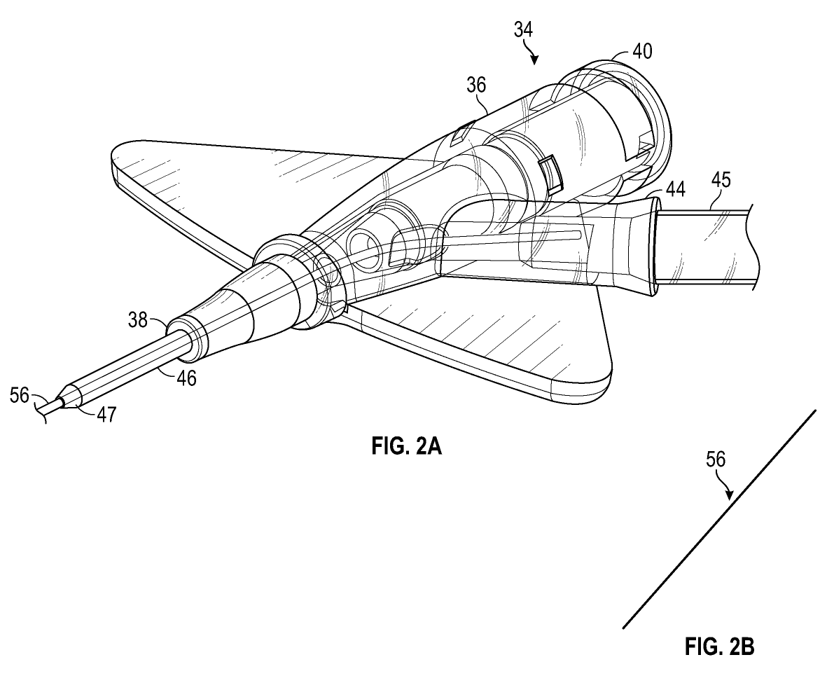

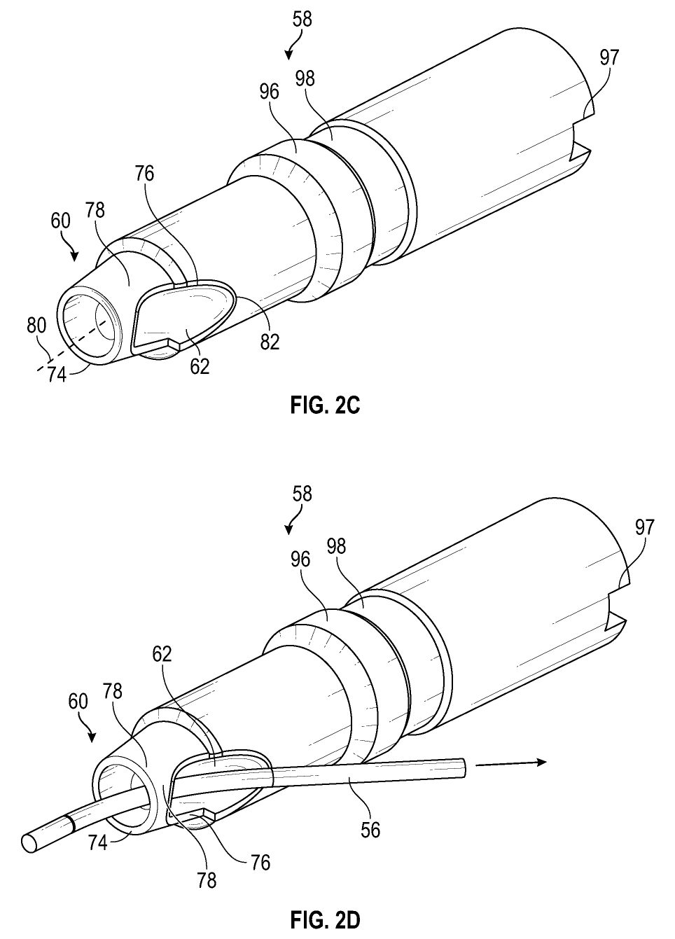

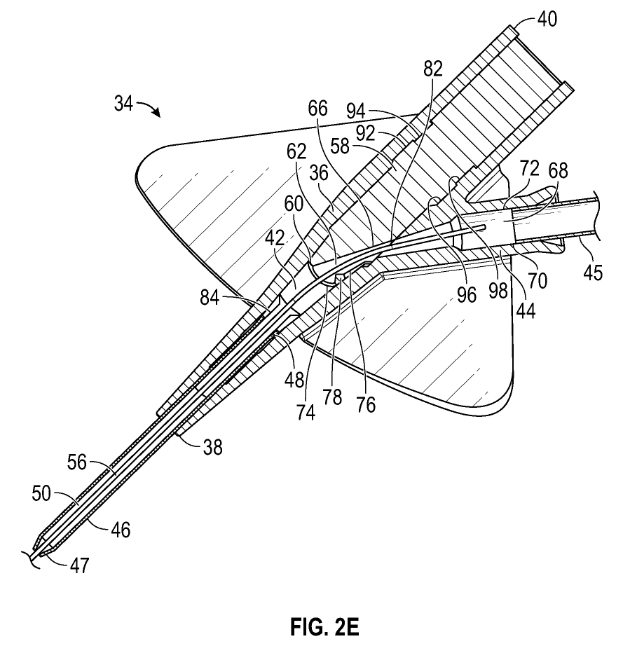

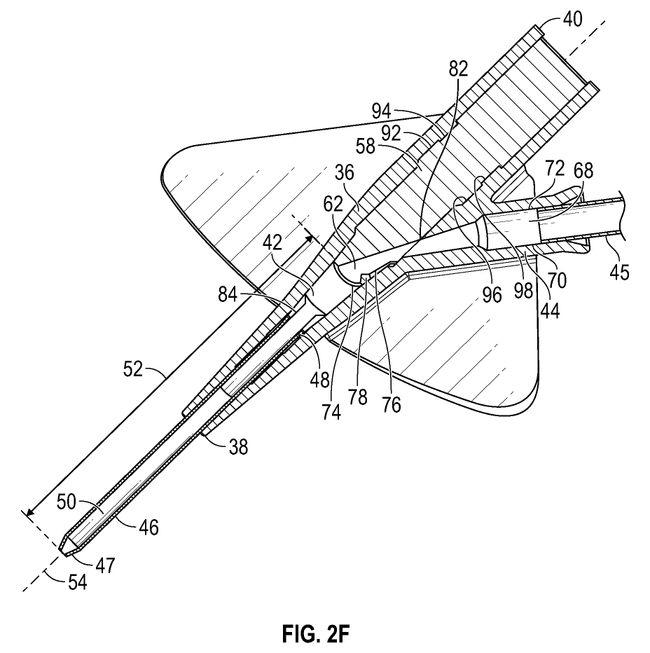

Referring now to FIGS. 2A-2F, in some embodiments, a catheter assembly 34 may include a catheter adapter 36, which may include a distal end 38, a proximal end 40, an inner surface forming a lumen 42 extending between the distal end 38 of the catheter adapter 36 and the proximal end 40 of the catheter adapter 36, and a side port 44 disposed between the distal end 38 of the catheter adapter 36 and the proximal end 40 of the catheter adapter 36. In some embodiments, the catheter assembly 34 may include an integrated IV catheter assembly having an extension tubing 45 integrated with the side port 44 and enabling collection of blood therethrough.

In some embodiments, the catheter assembly 34 may include a catheter 46 extending from the distal end 38 of the catheter adapter 36. In some embodiments, the catheter 46 may include a distal end 47, a proximal end 48, and a lumen 50 extending between the distal end 47 of the catheter 46 and the proximal end 48 of the catheter 46. Non-limiting examples of the catheter 46 may include a peripheral intravenous catheter (PIVC) or an arterial catheter for insertion into a vein and an artery, respectively, of the patient.

In some embodiments, the lumen 50 of the catheter 46 and the lumen 50 of the catheter adapter 36 may include an open pathway 52 extending along an axis 54. In some embodiments, the axis 54 may correspond to a longitudinal axis or central axis of the catheter assembly 34 and/or the lumen 42. In some embodiments, the open pathway 52 may be configured to receive a guidewire 56 when the catheter assembly 34 is threaded distally over the guidewire 56. In some embodiments, the open pathway 52 may be unobstructed allowing a straight object, such as the guidewire 56, to be received therein.

In some embodiments, the catheter assembly 34 may include a redirection component 58 disposed within the lumen 42 of the catheter adapter 36 and sealing the lumen 42 of the catheter adapter 36 proximal to the side port 44. In some embodiments, the redirection component 58 may facilitate use of the Seldinger technique outlined in FIGS. 1A-1E or a similar technique because the guidewire 56 may be guided or redirected to the side port 44 from the open pathway 52 or the axis 54 when the catheter assembly 34 is threaded on the guidewire 56. FIG. 2A illustrates the catheter adapter 36 as transparent for illustrative purposes, and the catheter assembly 34 may be threaded further over the guidewire 56 than illustrated in FIG. 2A such that the guidewire 56 can be removed from the clinician through the side port 44.

In the prior art illustrated in FIG. 1F, during use of the Seldinger technique, the guidewire 56 would contact and/or enter the septum 32 and have difficulty being moved into the side port 28. Moreover, the guidewire 56 contacting and/or entering the septum 32 increases a risk of blood leakage out of the prior art catheter assembly as well as guidewire instability. In some embodiments, the redirection component 58 of the present disclosure may facilitate using the Seldinger technique with an integrated IV catheter assembly, allowing blood collection while also eliminating an over-the-needle catheter and a risk of inappropriate placement of the catheter due to the offset between the tip of the introducer needle and the start of the catheter.

In some embodiments, the guidewire 56 may include any one of a multitude of components useful in placing a catheter within the body of a patient. For example, the guidewire 56 may include a thin, flexible wire for placing a catheter. In some embodiments, the guidewire 56 may be straight or generally straight to easily travel through the open pathway 52 along the axis 54. In some embodiments, the guidewire 56 may be configured to bend or flex to an angle of the side port 44 in response to the catheter assembly 34 being threaded distally over the guidewire 56 and redirected to the side port 44 by the redirection component 58. In some embodiments, the guidewire 56 may be constructed of metallic guidewire elements or materials known in the art.

In some embodiments, the redirection component 58 may be monolithically formed as a single unit. In some embodiments, the lumen 42 does not include a septum because it is not needed due to the redirection component 58 sealing the lumen 42. In some embodiments, the redirection component 58 may permanently seal the lumen 42 of the catheter adapter 36 proximal to the side port 44. In further detail, in some embodiments, unlike a septum, the redirection component 58 may not open or close, which reduces a risk of blood leaking through the proximal end of the catheter adapter.

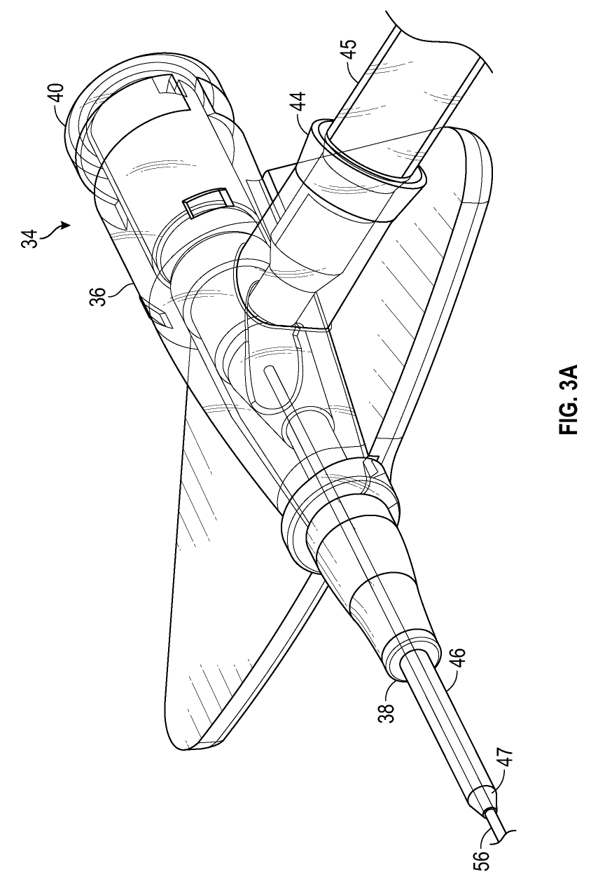

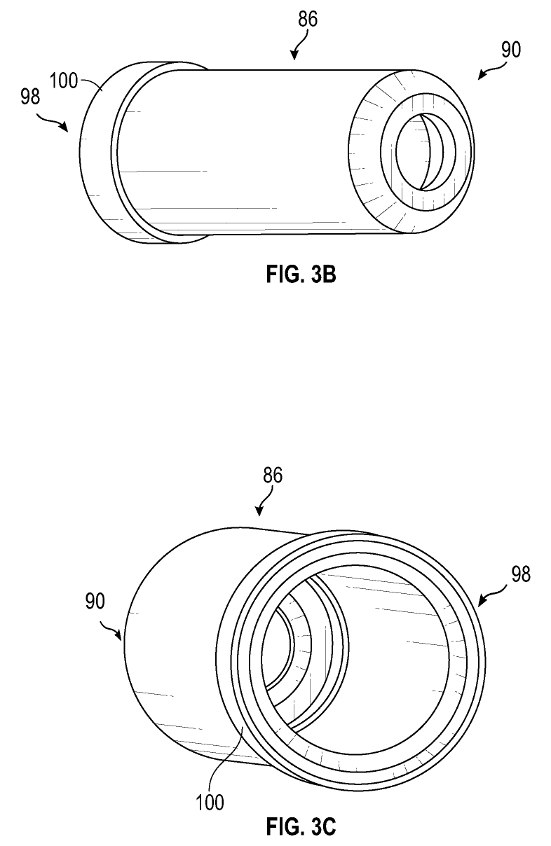

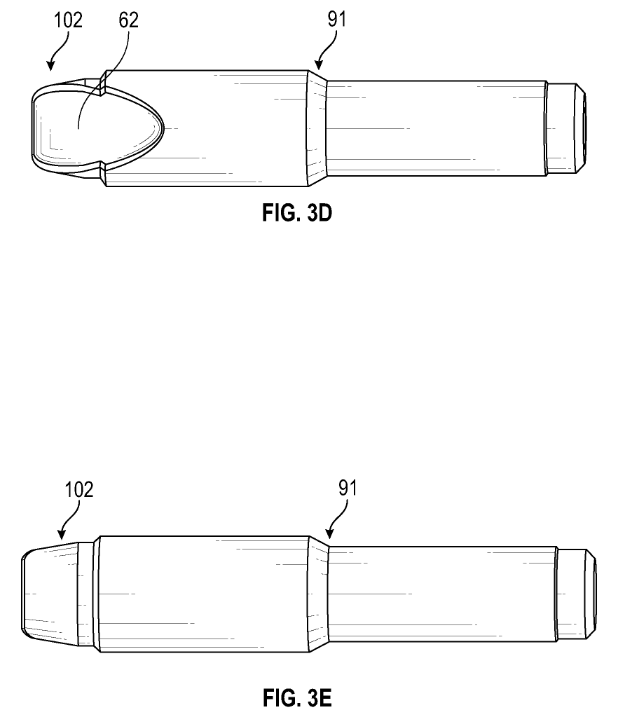

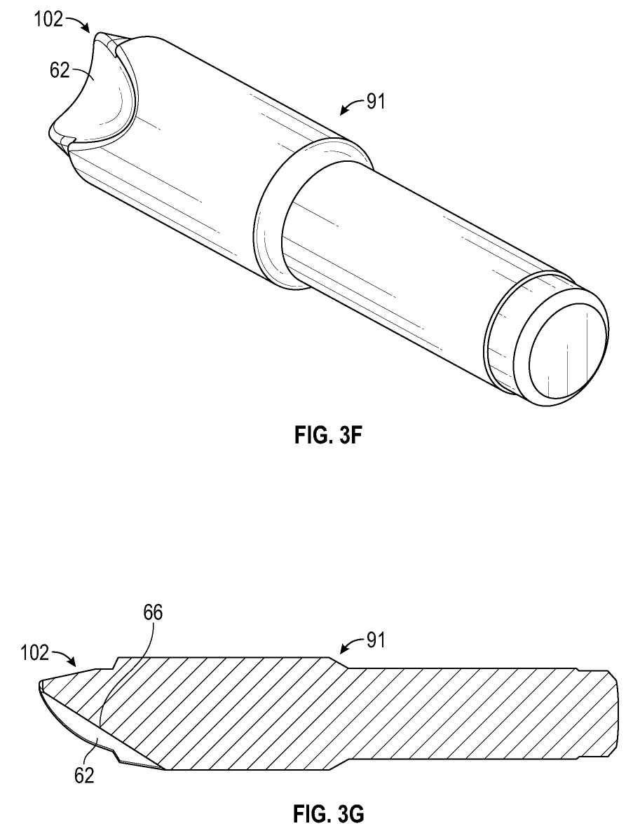

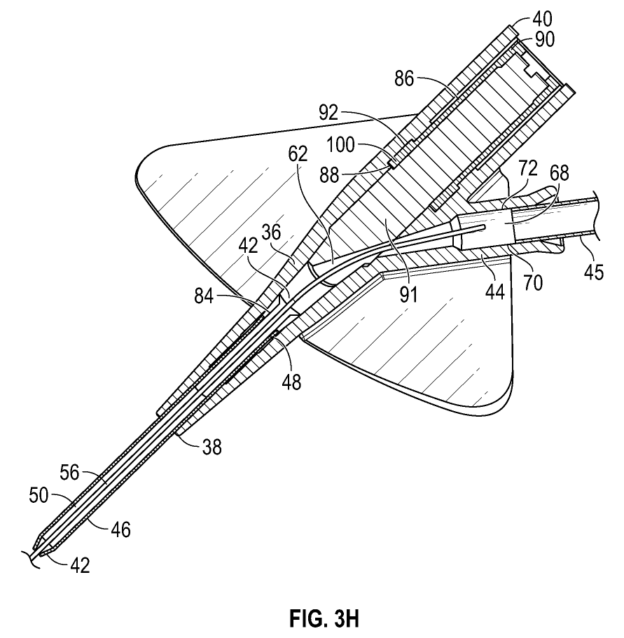

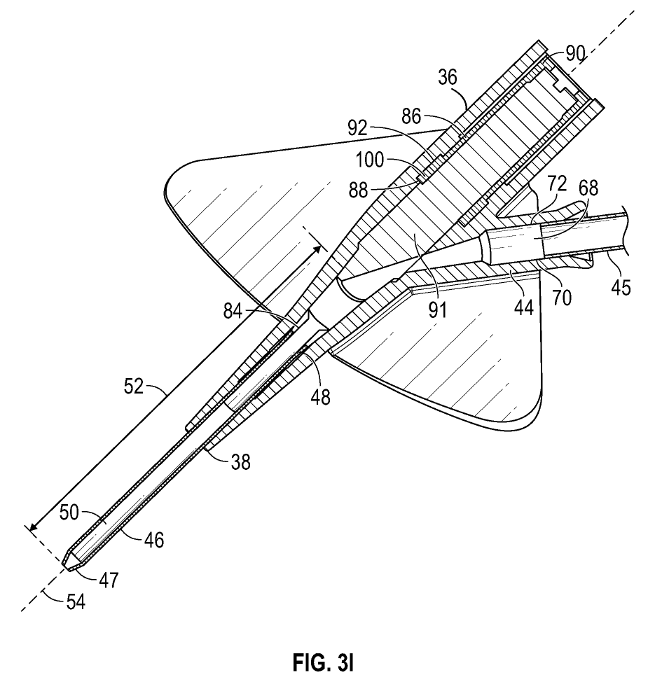

Moreover, in some embodiments, since the catheter assembly does not include an introducer needle or an over-the-needle catheter, a material of the redirection component 58 can vary since the introducer needle does not need to pass through the redirection component 58. For example, the redirection component 58 may be constructed of one or more hard plastics, such as, for example, polycarbonate, which may prolong a life of the redirection component 58 and facilitate securement of the redirection component 58 within the catheter adapter 36. In some embodiments, the redirection component 58 may be constructed of one or more soft plastics, such as, for example, polyisoprene or silicone, which may reduce wear on the guidewire 56. In some embodiments, the redirection component 58 may be constructed of a first material and a canister may be constructed of a second material more rigid than the first material, as described further with respect to FIG. 3. This hybrid approach may achieve both objectives of securement of the redirection component 58 within the catheter adapter 36 and reducing wear on the guidewire 56.

In some embodiments, the redirection component 58 may include a distal end 60 having a surface 62 disposed at a proximal end of the open pathway 52 to block the open pathway 52. Thus, in some embodiments, the open pathway 52 may extend to the surface 62 or to a portion of the surface 62 aligned with the axis 54. In some embodiments, the surface 62 may be configured to redirect the guidewire 56 from the axis 54 into the side port 44 when the catheter assembly 34 is threaded distally over the guidewire 56. In some embodiments, the side port 44 may be angled with respect to the axis 54.

In some embodiments, an angle of the side port 44 with respect to the axis 54 or a longitudinal axis of the catheter adapter 36 may vary. In some embodiments, to facilitate blood flow therethrough, the side port 44 may be angled between 45˚ and 90˚, inclusive, with respect to the axis 54 and oriented in a proximal direction. In some embodiments, the redirection component 58 may facilitate positioning of the side port 44 at angles greater than 45˚ with respect to the axis 54 or a longitudinal axis of the catheter adapter 36 (such as up to and including 90˚) because the redirection component 58 may facilitate entry of the guidewire 56 into the side port 44 when the catheter assembly 34 is threaded over the guidewire 56.

As illustrated in FIGS. 2C-2F, in some embodiments, the surface 62 may include a groove, which may include a rounded trough shape. In these and other embodiments, a cross-section of the groove 64 may include a linear portion 66 angled with respect to the axis 54. In some embodiments, the linear portion 66 may divide the rounded trough shape in half. In some embodiments, an angle of the linear portion 66 with respect to the axis 54 or the longitudinal axis of the catheter adapter 36 may vary in response to the angle of the side port 44 with respect to the longitudinal axis of the catheter adapter 36. In further detail, in some embodiments, the angle of the linear portion with respect to the longitudinal axis of the catheter adapter 36 may increase in response to an increased angle of the side port 44 with respect to the longitudinal axis of the catheter adapter 36.

In some embodiments, the side port 44 may include an inner surface forming a side port lumen 68. In some embodiments, a cross-section of the side port 44 may include a distal wall 70 and a proximal wall 72 opposite the distal wall 70. In some embodiments, the surface 62 may be distal to or aligned with the proximal wall 72 of the inner surface of the side port 44, which may prevent blood from flowing proximally to the side port lumen 68 and/or getting caught proximal to the side port lumen 68.

In some embodiments, the side port 44 may include the inner surface forming the side port lumen 68, and the distal end 60 of the redirection component 58 may include a first opening 74 and a second opening 76 separated from the first opening 74 by an arm 78. In some embodiments, when the catheter assembly 34 is threaded distally over the guidewire 56, the first opening 74 may move over the guidewire 56 before the second opening 76. In some embodiments, a central axis 80 of the first opening 74 may be aligned with the axis 54. In some embodiments, the first opening 74 may be circular and configured to center the guidewire 56. In some embodiments, a proximal end 82 of the second opening 76 may be distal to or aligned with the proximal wall 72 of the inner surface of the side port 44, which may prevent blood from flowing proximally to the side port lumen 68 and/or getting caught proximal to the side port lumen 68. In some embodiments, the second opening 76 may be elongated and/or configured to guide the guidewire 56 to the side port 44.

In some embodiments, the redirection component 58 may extend distal to at least the proximal wall 72 of the side port 44 such that the redirection component 58 removes space within the lumen 42. In some embodiments, the redirection component 58 and/or one or more components thereof (such as the first opening 74, the second opening 76, and the arm 78) may improve flushing of the catheter assembly 34 by removing space within the lumen 42 and creating a direct fluid path that is easily flushed. In some embodiments, the redirection component 58 and/or one or more components thereof (such as the first opening 74, the second opening 76, and the arm 78) may improve reduce a risk of air bubbles entering the body of the patient by removing space within the lumen 42 and creating a direct fluid path that is easily primed.

In some embodiments, the catheter assembly 34 may include a wedge 84 disposed within the lumen 42 of the catheter adapter 36 and securing the catheter 46 within the catheter adapter 36. In some embodiments, the open pathway 52 may extend through the wedge 84.

In some embodiments, the inner surface of the catheter adapter 36 may include an annular groove 92 and/or an annular protrusion 94 proximate or immediately adjacent the annular groove 92. In some embodiments, the redirection component 58 may include an outer surface having an annular protrusion 96 configured to lock the redirection component 58 within the annular groove 92. In some embodiments, the outer surface of the redirection component 58 may also include an annular recess 98 proximate or immediately adjacent the annular protrusion 96 and configured to receive the annular protrusion 94. In some embodiments, the annular protrusion 96 and/or the annular recess 98 may facilitate securement of the redirection component 58 within the catheter adapter 36 in a snap fit.

In some embodiments, a proximal end of the redirection component 58 may include an alignment feature 97 such as, for example, an alignment notch that may facilitate orientation of the redirection component 58 within the lumen 42 through alignment with one or more features of the catheter adapter 36.

Referring now to FIGS. 3A-3I, in some embodiments, the catheter assembly 34 may include a canister 86 having a distal end 88 and a proximal end 90 and disposed within the lumen 42 of the catheter adapter 36. In some embodiments, a redirection component 91 may be secured within the canister 86. In some embodiments, the redirection component 91 may be similar or identical to the redirection component 58 of FIGS. 2A and 2C-2E in terms of one or more features and/or operation. In some embodiments, an outer diameter of the redirection component 91 may be slightly less than an inner diameter of the canister 86 such that the redirection component 91 fits snugly within the canister 86. In some embodiments, the redirection component 91 may include an elastic or flexible material. In these embodiments, the outer diameter of the redirection component 91 may be slightly greater than than the inner diameter of the canister 86 such that the redirection component 91 is slightly compressed within the canister 86. In some embodiments, the canister 86 may be secured to the catheter adapter 36.

In some embodiments, the redirection component 58 may be constructed of a first material, and the canister 86 may be constructed of a second material. In some embodiments, the second material may be rigid. In some embodiments, the first material may be less rigid than the second material, which may achieve both objectives of securement of the redirection component 58 within the catheter adapter 36 and reducing wear on the guidewire 56. In some embodiments, the first material may be flexible. In some embodiments, the first material may include polyisoprene or silicone.

In some embodiments, the inner surface of the catheter adapter 36 may include the annular groove 92. In some embodiments, the canister 86 may include an outer surface having an annular protrusion 100 configured to lock or snap the canister 86 within the annular groove 92.

In some embodiments, the redirection component 91 may include one or more of the first opening 74, the second opening 76, and the arm 78, as illustrated clearly in FIGS. 2C-2D. In other embodiments, a distal end 102 of the redirection component 91 may not include the first opening 74, the second opening 76, and the arm 78.

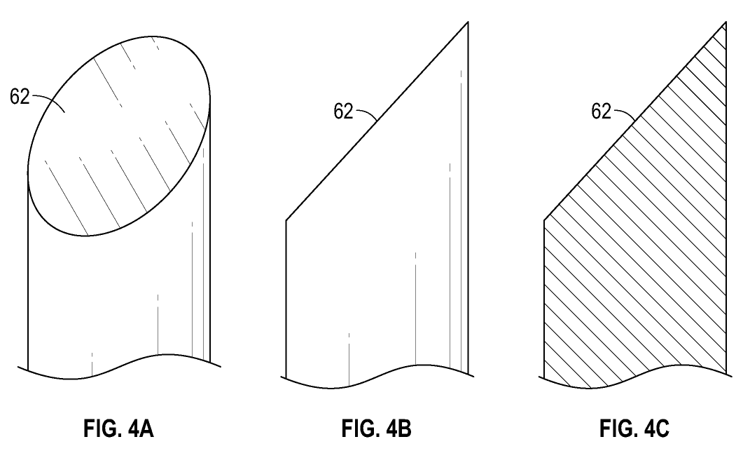

Referring now to FIGS. 4A-4C, in these embodiments, the surface 62 of the redirection component 58 of FIG. 2 and/or the redirection component 91 of FIG. 3 may be angled with respect to the axis 54 and planar. In some embodiments, the surface 62 that is planar may extend across a lumen of the catheter adapter to guide a guidewire exiting the wedge 84 (see FIGS. 2E or 3H, for example) into the side port 44 (see FIGS. 2E or 3H, for example).

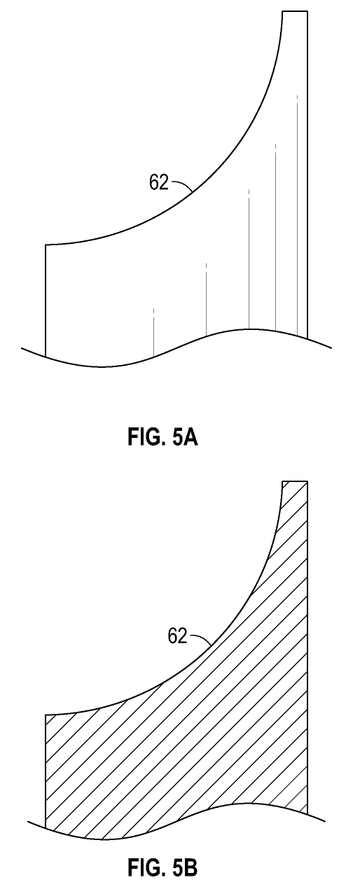

Referring now to FIGS. 5A-5B, in some embodiments, the surface 62 of the redirection component 58 of FIG. 2 and/or the redirection component 91 of FIG. 3 may be curved or arc-shaped. In some embodiments, a curve or an arc shape of the surface 62 may extend across a lumen of the catheter adapter to guide a guidewire exiting the wedge 84 (see FIGS. 2E or 3H, for example) into the side port 44 (see FIGS. 2E or 3H, for example).

All examples and conditional language recited herein are intended for pedagogical objects to aid the reader in understanding the present disclosure and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the present disclosure.

What is claimed:

1. A catheter assembly, comprising:
a catheter adapter, comprising a distal end, a proximal end, an inner surface forming a lumen extending between the distal end of the catheter adapter and the proximal end of the catheter adapter, and a side port disposed between the distal end of the catheter adapter and the proximal end of the catheter adapter;
a catheter extending from the distal end of the catheter adapter, wherein the catheter comprises a distal end, a proximal end, and a lumen extending between the distal end of the catheter and the proximal end of the catheter, wherein the lumen of the catheter and the lumen of the catheter adapter comprise an open pathway extending along an axis, wherein the open pathway is configured to receive a guidewire when the catheter assembly is threaded distally over the guidewire; and
a redirection component disposed within the lumen of the catheter adapter and sealing the lumen of the catheter adapter proximal to the side port, wherein the redirection component comprises a distal end having a surface disposed at a proximal end of the open pathway to block the open pathway, wherein the surface is configured to redirect the guidewire from the axis into the side port when the catheter assembly is threaded distally over the guidewire, wherein the side port is angled with respect to the axis.

2. The catheter assembly of claim 1, wherein the surface comprises a groove, wherein a cross-section of the groove comprises a linear portion angled with respect to the axis.

3. The catheter assembly of claim 1, wherein the surface is angled with respect to the axis and planar.

4. The catheter assembly of claim 1, wherein the surface is arc-shaped.

5. The catheter assembly of claim 1, further comprising a canister disposed within the lumen of the catheter adapter, wherein the redirection component is secured within the canister, wherein the canister is secured to the catheter adapter, wherein the redirection component is constructed of a first material and the canister is constructed of a second material, wherein the first material less rigid than the second material.

6. The catheter assembly of claim 1, wherein the side port is angled between 45˚ and 90˚, inclusive, with respect to the axis.

7. The catheter assembly of claim 1, wherein the redirection component permanently seals the lumen of the catheter adapter proximal to the side port.

8. The catheter assembly of claim 1, wherein the inner surface of the catheter adapter comprises an annular groove, wherein the redirection component comprises an outer surface having an annular protrusion configured to lock the redirection component within the annular groove.

9. The catheter assembly of claim 1, wherein the side port comprises an inner surface forming a side port lumen, wherein the surface is distal to or aligned with a proximal wall of the inner surface of the side port.

10. The catheter assembly of claim 1, wherein the side port comprises an inner surface forming a side port lumen, wherein the distal end of the redirection component further comprises an first opening and a second opening separated from the first opening by an arm, wherein when the catheter assembly is threaded distally over the guidewire, the first opening moves over the guidewire before the second opening, wherein a central axis of the first opening is aligned with the axis, wherein a proximal end of the second opening is distal to or aligned with a proximal wall of the inner surface of the side port.

11. The catheter assembly of claim 1, further comprising a wedge disposed within the lumen of the catheter adapter and securing the catheter within the catheter adapter, wherein open pathway extends through the wedge.

12. A method of manufacturing, comprising:
providing a catheter adapter, wherein the catheter adapter comprises a distal end, a proximal end, an inner surface forming a lumen extending between the distal end of the catheter adapter and the proximal end of the catheter adapter, and a side port disposed between the distal end of the catheter adapter and the proximal end of the catheter adapter;
coupling a catheter to the catheter adapter, wherein the catheter extends from the distal end of the catheter adapter, wherein the catheter comprises a distal end, a proximal end, and a lumen extending between the distal end of the catheter and the proximal end of the catheter, wherein the lumen of the catheter and the lumen of the catheter adapter comprise an open pathway extending along an axis, wherein the open pathway is configured to receive a guidewire when the catheter assembly is threaded distally over the guidewire; and
positioning a redirection component within the lumen of the catheter adapter, wherein the redirection component seals the lumen of the catheter adapter proximal to the side port, wherein a distal end of the redirection component comprises a surface disposed at a proximal end of the open pathway to block the open pathway, wherein the surface is configured to redirect the guidewire from the axis into the side port when the catheter assembly is threaded distally over the guidewire, wherein the side port is angled with respect to the axis.

13. The method of claim 12, wherein the surface comprises a groove, wherein a cross-section of the groove comprises a linear surface angled with respect to the axis.

14. The method of claim 12, wherein the surface is angled with respect to the axis and planar.

15. The method of claim 12, wherein the surface is arc-shaped.

16. The method of claim 12, further comprising a canister disposed within the lumen of the catheter adapter, wherein the redirection component is secured within the canister, wherein the canister is secured to the catheter adapter, wherein the redirection component is constructed of a first material and the canister is constructed of a second material, wherein the first material less rigid than the second material.

17. The method of claim 12, wherein the side port is angled between 45˚ and 90˚, inclusive, with respect to the axis.

18. The method of claim 12, wherein the redirection component permanently seals the lumen of the catheter adapter proximal to the side port.

19. The method of claim 12, wherein the inner surface of the catheter adapter comprises an annular groove, wherein the redirection component comprises an outer surface having an annular protrusion configured to lock the redirection component within the annular groove.

20. The method of claim 12, wherein the side port comprises an inner surface forming a side port lumen, wherein the surface is distal to or aligned with a proximal wall of the inner surface of the side port.
